(19) United States
(12) Patent Application Publication
(10) Pub. No.: US 2002/0049167 A1
(43) Pub. Date: Apr. 25, 2002

(54) Azithromycin as a therapeutic agent for infections with leishmania parasites

(76) Inventors: David Abraham, Wynnewood, PA (US); Alejandro Javier Kroleweicki, Buenos Aires (AR)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 09/813,374

(22) Filed: Mar. 21, 2001

Related U.S. Application Data
(60) Provisional application No. 60/191,264, filed on Mar. 22, 2000.

(51) Int. Cl. A61K 31/7052; C12Q 1/18

(52) U.S. Cl. 514/28; 435/32

(57) ABSTRACT

The present invention generally relates to an in vitro analysis of anti-Leishmania activity of the anti-parasitic agent azithromycin, and more particularly the invention uses a novel source of macrophages, which are monocyte-derived bone marrow macrophages, to show the efficacy of azithromycin anti-parasitic therapy.

### CROSS REFERENCE TO RELATED APPLICATIONS

This application claims priority under 35 U.S.C. §119 based upon U.S. Provisional Patent Application No. 60/191264 filed Mar. 22, 2000.

### FIELD OF THE INVENTION

The present invention relates to an improved method for treating Leishmaniasis, specifically treating patients infected with Leishmania sp. with the therapeutic agent azithromycin.

### BACKGROUND OF THE INVENTION

Parasites of the genus Leishmania are protozoans that cause mammalian disease. Leishmania sp. are widely distributed in tropical and subtropical regions and are transmitted by the bites of sandflies. Parasitic infections are manifested in cutaneous, mucosal and/or visceral symptomologies.

Currently, the risk for Leishmania infection is significant, covering an estimated population of 350 million people (Desjeux P. Leishmaniasis. Public health aspects and control. Clinics in Dermatology. 14(5):417-23, 1996). Leishmaniasis occurs in one to two million people annually and is, therefore, considered a priority by the World Health Organization. In addition to human infections, Leishmania infections are of significant concern in dogs and other mammals. The visceral form (Kala-azar) accounts for approximately half a million infections (Badaro R., et al., Leishmaniose visceral. In Veronesi, Focaccia, eds. Tratado de Infectologia. São Paulo. Atheneu Edit. 1997. 1234-58). In addition to the infections endemic in tropical climates, leishmaniasis is emerging as an opportunistic infection in the acquired immunodeficiency syndrome (AIDS) population, thereby increasing the urgency for the development of an effective, and safe, therapeutic regimen. (Alvar J. et al., Clinics in Dermatology. 14(5):541-6, 1996). The burden that this disease has placed on society has caused a strong interest in finding new therapeutic options to replace those currently available. (Berman JD. Clinical Infectious Diseases. 24(4): 684-703, 1997).

Therapies against human leishmaniasis include pentavalent antimonials (sodium stibogluconate and meglumine antimonate), amphotericin B and some of its new lipid formulations and pentamidine (Berman JD. Clinical Infectious Diseases. 24(4):684-703, 1997; Berman J. Current Opinion in Infectious Diseases. 11(6): 707-710, 1998). Paromomycin, an aminoglycoside, has also shown anti-Leishmania activity, but few patients have been treated and the efficacy has been variable in different areas of the world where it was studied (Berman J. Current Opinion in Infectious Diseases. 11(6):707-710, 1998). These drugs have several disadvantages: 1) their cost is prohibitively high; 2) they are unavailable for oral administration (some of them like amphotericin B can only be used intravenously); and/or 3) they may cause serious side effects that require close monitoring of the patients (Berman JD. Clinical Infectious Diseases. 24(4):684-703, 1997). These elements make the approach to leishmaniasis very problematic, and in many cases this results in the inability to provide effective treatment. Although currently there are available therapeutics to treat the human form of leishmaniasis, treatments for dogs and other mammals are unavailable. Ideally, an optimal therapeutic agent for a pharmaceutical composition for treating leishmaniasis is effective against macrophage-dwelling amastigotes, is available in an oral prescription, is tolerated without toxic side effects and has no contraindications in children and pregnant women.

Azithromycin is an azalide antibiotic that belongs to the family of macrolides. Characteristics that make it a very good candidate for anti-Leishmania activity include: 1) an administration route that is oral, intravenous or by injection; 2) good oral bioavailability and a long half life (Lalak N.J. Morris DL. Clinical Pharmacokinetics. 25(5):370-4, 1993; Foulds G. et al., Journal of Antimicrobial Chemotherapy. 25 Suppl A:73-82, 1990); 3) drug accumulation in the tissue cells, especially in macrophages, of levels reaching 110 times greater than that obtained in the serum (Gladue RP. et al., Antimicrobial Agents & Chemotherapy. 33(3):277-82, 1989); 4) there are no contraindications for use in children and pregnant women (FDA category B); and 5) there are few, if any, toxic side effects (Steigbigel N. Macrolides and clindamycin. In Mandell G, Bennett J, Dolin R, eds. Mandell, Douglas and Bennett's Principles and practice of infectious diseases. 4^{th} ed. 1995. 334-46).

Several different protozoan infections of humans have been shown in vitro and in vivo to be susceptible to azithromycin in variable degrees. These include Acanthamoeba sp (Schuster, F. L. and Visvesvara, G. S., Journal of Eukaryotic Microbiology, 45(6): 612-618, 1998), Cryptosporidium parvum (Hicks, P., et al., Journal of Pediatrics, 129(2): 297-300, 1996), Plasmodium falciparum, Plasmodium malaria, Plasmodium vivax (Taylor, W.R., et al., Clinical Infectious Disease, 28(1): 74-81, 1999; Sadiq, S. T., et al., Lancet, 346(8979): 881-2, 1995; Andersen, SL., et al., American Journal of Tropical Medicine & Hygiene, 52(2): 159-60, 1995) and Toxoplasma gondii. Studies involving Toxoplasma gondii and Cryptosporidium parvum have demonstrated direct effects of the drug on the parasite's viability by blocking protein synthesis. (Beckers, C. J., et al., Journal of Clinical Investigation, 95(1): 367-76, 1995; Dumas J L. et al., Journal of Antimicrobial Chemotherapy. 34(1):111-8, 1994; Dupont C. et al., Journal of Clinical Microbiology. 34(1):227-9, 1996). Studies involving Toxoplasma and the subcellular mechanisms of susceptibility to this drug, have shown that azithromycin concentrates in the lysosomes of the infected macrophages (Schwab J C. et al., Antimicrobial Agents & Chemotherapy. 38(7): 1620-7, 1994., Beckers CJ. et al.,. Journal of Clinical Investigation. 95(1): 367-76, 1995); therefore, a site of maximal drug concentration would be the phago-lysosome where Leishmania resides inside the macrophage, thereby supporting the efficacy of azithromycin against these infections. In addition, recent publications have demonstrated the therapeutic and prophylactic efficacy of azithromycin against malarial parasites (Taylor WRJ. et al., Clinical Infectious Diseases. 28(1):74-81, 1999; Sadiq ST. et al., Lancet. 346(8979):881-2, 1995).

Macrolide antibiotics exert immunomodulatory effects that are thought to be due to the high intra-cellular accumulation of the drug in white blood cells (Khan AA et al., International Journal of Antimicrobial Agents. 11(2): 121-32, 1999). This group of antibiotics was shown to activate macrophages and increase the cytocidal activity of macrophages against Candida albicans, as well as stimulating IL-2 secretion (Xu G. et al., Microbiology & Immunology. 40(7): 473-9, 1996). This stimulation of macrophage activity by macrolide antibiotics will cause an additive effect in the anti-parasite activity of the drug.

The present invention discloses assays to test the susceptibility of Leishmania to different drugs in vitro. The assays described herein use a novel source of macrophages as host cells for Leishmania: monocyte-derived bone marrow macrophages. The advantage of using these cells, rather than the more commonly used peritoneal macrophages, resides in the higher yield of cells per mouse (50 million vs. 0.7 to 1.2 million cells per mouse). Previous studies have demonstrated variable results in determining the susceptibility of Leishmania sp. to different drugs dependent on the source of the macrophages. Specifically, it has been shown that Leishmania sp. were susceptible to pentavalent antimony in vivo and in vitro in mouse peritoneal macrophages and in human monocyte-derived macrophages but not in tumor macrophages (Berman, J D. & Wyler D J. Journal of Infectious Diseases, 142(1): 83-6, 1980., Mattock, N M. & Peters W. Annals of Tropical Medicine & Parasitology. 69(3): 359-71, 1975., Neal, RA. & . Matthews P J. Transactions of the Royal Society of Tropical Medicine & Hygiene, 76(2): 284, 1982.). Monocyte-derived bone marrow macrophages are, therefore, shown herein to be an alternative source of cells for in vitro testing of drugs for efficacy against Leishmania sp.

The infection levels were measured by microscopic observation of the macrophages to determine the number of infected cells and the number of parasites found per cell. Newer assays measure radioactive material uptake by infected macrophages; these assays have the advantage of lower variability, mechanical counting and a more accurate determination of parasite viability; however, they require more sophisticated equipment and have similar results when compared with the old method (Berman JD. Journal of Parasitology. 70(4):561-2, 1984).

### SUMMARY OF THE INVENTION

The present invention relates to an anti-parasitic macrolide antibiotic, preferably an azalide and more preferably an azithromycin. The present invention further describes the anti-parasitic activity of the antibiotic against a Leishmania sp., which is effective in any mammal, preferably humans and dogs. It is a further object of the present invention that Leishmania sp. is endemic to a tropical and sub-tropical climate.

It is another object of the present invention that the anti-parasitic effect of the antibiotic is effective toward a cutaneous, mucosal or visceral manifestation of leishmaniasis. It is a further object that the antibiotic is effective toward an opportunistic infection, preferably in patients with acquired immunodeficiency syndrome (AIDS)

It is another object of the present invention that azithromycin is a pharmaceutical composition for treating leishmaniasis and is effective against macrophage-dwelling amastigotes.

It is a further object that the azithromycin pharmaceutical composition of the present invention is administered as an oral or injectable prescription, preferably an oral prescription. In one embodiment the azithromycin pharmaceutical composition is bioavailable and has a long half life. Additionally, the azithromycin pharmaceutical composition is tolerated without toxic side effects and is a safe prescription in children and pregnant women.

It is another object of the present invention that the azithromycin pharmaceutical composition accumulates in tissue cells, preferably macrophages. In a specific embodiment the accumulation is at a site of maximal azithromycin concentration in a phago-lysosome inside the macrophage. It is a further object that the concentration of azithromycin in the phago-lysosome exerts an immunomodulatory effect. In one embodiment the immunomodulatory effect is caused by the activation of macrophages. The activation is caused by an increased cytocidal activity of or a stimulation of IL-2 secretion by the macrophages.

It is a further object of the present invention that the azithromycin induced immunomodulatory effect on macrophages is additive to the direct killing of the parasite by azithromycin.

The present invention further discloses a method of testing the in vitro susceptibility of Leishmania sp. to drugs, by culturing amastigotes with monocyte-derived bone marrow macrophages, adding concentrations of an antibiotic to be tested, and measuring the levels of macrophage infection, wherein a number of infected macrophages and a number of parasites in the infected macrophages are counted. In one embodiment the drug tested in the method is azithromycin.

### BRIEF DESCRIPTION OF THE DRAWINGS

FIG. 1. Multiplication Index for Leishmania major in murine macrophages placed in in vitro culture with 0.1-15 μg/ml of azithromycin or 1 mg/ml of amphotericin B. Multiplication index was calculated as [mean number of amastigotes per 100 macrophages in experimental cultures/mean number of amastigotes per 100 macrophages in control cultures]×100. *=values are statistically different from 0.1 μg/ml of azithromycin (P<0.05). **=values are statistically different from 1 mg/ml of amphotericin B(P<0.05).

### DETAILED DESCRIPTION OF THE INVENTION

Methods

Macrophages

Monocyte-derived bone marrow macrophages from C57B1/6 naïve mice. These cells were harvested from the femoral bones and incubated for 7 days at 37° C. in 5% CO₂ in complete tissue culture medium [DMEM, 20% heat inactivated fetal calf serum, 1% Penicillin/streptomycin, 1% glutamine, 1% pyruvate, 30% L-cell line supernatant]. After 7 days, 50×10⁶ macrophages per mouse donor were recovered from the cultures. The cells were then washed and 1×10⁶ macrophages were added to the wells (16 mm diameter) of a 24 well plate at the bottom of which was placed a round glass cover slip. The macrophages were added to the wells in 300 μl of culture medium consisting of RPMI 1640,10% heat inactivated fetal calf serum, 1% Penicillin/streptomycin, 1% glutamine, 2.5% BEPES, and 0.1% 2-mercaptoethanol.

Parasites

Cryopreserved amastigotes of Leishmania major (Friedlin strain), originally obtained from footpad lesions of SCID mice, were thawed, washed with medium and counted in a hemocytometer, then re-suspended in complete medium and added to the wells at a concentration of 1 million per well (amastigote/macrophage ratio 1:1) in 50 μl of complete medium. The total volume of media in each well was 35 μl.

Antibiotics

Amphotericin B (Fungizone, Gibco) was reconstituted from a lyophilized form and azithromycin (Zithromax, Pfizer) was prepared from a 500 mg-powder vial for IV use; both antibiotics were reconstituted in sterile water and diluted in complete medium to the desired concentration. Amphotericin B was used at a concentration of 1 mg/ml and azithromycin at concentrations of 0.1, 0.6, 3 and 15 μg/ml.

After mixing the macrophages with the amastigotes, the plates were incubated for 3 hrs. at 37° C. in 5% Co₂; at that point the plates were washed with PBS to eliminate non-adherent macrophages and free amastigotes. Medium with or without antibiotics was added to the wells at a final volume of 3 ml. Some of the cultures were removed from the wells at this point and placed in petri dishes containing 50 mls of medium with antibiotics. Amphotericin B was used as a positive control group in all the experiments. The cultures were maintained at 37° C. in 5% CO₂ and culture media and antibiotics were changed daily.

Before adding antibiotics on day 0 and on days 3 and 5, cover slips were recovered from the wells and petri dishes, immediately fixed and stained with Giemsa for counting, which was performed using 1000X magnification with immersion oil. All the experiments were carried out in duplicate.

Two cover slips were recovered from each time point and the data obtained for each slide was as follows: 200 to 300 macrophages were counted, number of infected macrophages per 100 macrophages and number of amastigotes per 100 macrophages was determined. The results reported are the mean values of those two parasite counts. Experiments were repeated a minimum of two times.

Results

Three mice were used as macrophage donors. The isolated cells were incubated for 7 days, yielding 150 million macrophages (50 million per mouse). The initial infection at the 1:1 amastigote per macrophage ratio rendered an initial infection of 27% amastigotes.

The effect of the antibiotics azithromycin and amphotericin B on L. major in vitro was measured using two parameters, the percentage of macrophages that were infected and the number of amastigotes that infected those macrophages. Infections of control cells were observed to approximately double after five days in culture both in terms of the number of cells infected and in terms of the number of amastigotes infecting those cells. A significant decrease was seen by day five in the percentage of macrophages infected in cultures inoculated with 0.6-15 μg/ml of azithromycin and with 1 mg/ml of amphotericin B. Furthermore, at this same time point, there was a significant decrease in the number of amastigotes found in those infected cells in cultures inoculated with 3.0-15 μg/ml of azithromycin and with 1 mg/ml of amphotericin B. Inoculation of the cultures with amphotericin B was more effective at controlling infection and replication of L. major than azithromycin at the concentrations tested. In all experiments, the morphology of the macrophages infected with L. major for five days improved as the concentration of azithromycin increased, with macrophages from cultures inoculated with 15 μg/ml having appearance comparable to those cultures exposed to amphotericin B or uninfected cells (Table 1).

TABLE 1
Effect of doses of azithromycin ranging from 0.1-15 μg/ml and
amphotericin B at 1 mg/ml on the ability of Leishmania major to infect
and multiply in murine macrophages in vitro for up to 5 days. Results
presented are: (1) the total number of parasites found per 100 macrophages
and (2) the percentage of macrophages infected without regard to the num-
ber of parasites found per cell. Numbers in bold represent 50% or greater
reduction from control.

| | Percent Infected | | | Number of Amastigotes/ | | |
|---|---|---|---|---|---|---|
| | Macrophages | | | 100 macrophages | | |
| Treatment | Day −0 | Day −3 | Day −5 | Day −0 | Day −3 | Day −5 |
| Control | 16 | 23 | 29 | 27 | 35 | 47 |
| 0.1 μg | 16 | 8 | 24 | 27 | 10 | 38 |
| azithromycin | | | | | | |
| 0.6 μg | 16 | 28 | 17 | 27 | 14 | 11 |
| azithromycin | | | | | | |
| 3.0 μg | 16 | 31 | 9 | 27 | 45 | 13 |
| azithromycin | | | | | | |
| 15 μg | 16 | 10 | 9 | 27 | 13 | 12 |
| azithromycin | | | | | | |
| Amphoteri- | 16 | 1 | 0 | 27 | 1 | 0 |
| cin B | | | | | | |

The activity of 0.6 and 3.0 μg/ml of azithromycin was then compared in two systems. The first system was 16 mm diameter wells containing a volume of 3 ml and the second, a Petri-dish containing a volume of 50 ml. The results of these experiments showed that after five days in culture 3.0 μg/ml of azithromycin prevented both parasite infection of cells and parasite replication regardless of the culture conditions. A difference was seen, however, in cultures containing 0.6 mg/ml of azithromycin. In the system with the higher volume of media and thus a higher net quantity of drug there was a decrease in parasite growth and infection. The number of parasites per cell and the percentage of cells infected were equivalent in the high volume system regardless of whether 0.6 or 3.0 μg/ml of azithromycin was used, thus confirming the ability of the macrophages to concentrate the drug to achieve active dose levels.

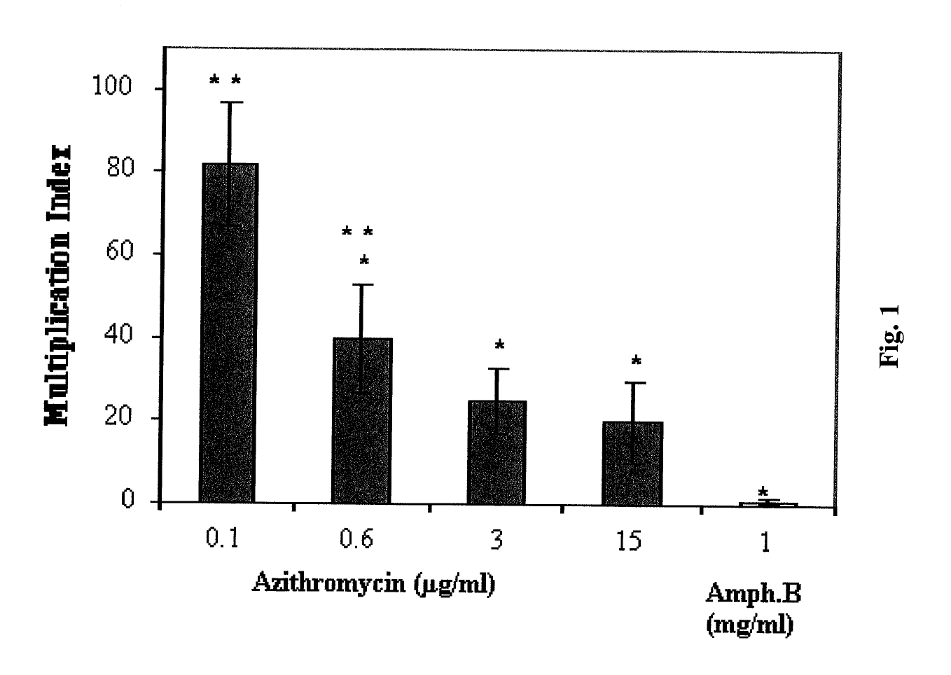

Finally, statistical analysis of the data was performed using a method to normalize the data between experiments. A multiplication index was calculated as [mean number of amastigotes per 100 macrophages in experimental cultures/mean number of amastigotes per 100 macrophages in control cultures]×100 for L. major in murine macrophages placed in in vitro culture with 0.1-15 μg/ml of azithromycin or 1 mg/ml of amphotericin B. The data was then analyzed by the statistical test analysis of variance and the results demonstrated that exposure of the parasite infected macrophages to 0.6-15 μg/ml of azithromycin or 1 mg/ml of amphotericin B caused a significant reduction in the multiplication index. Furthermore, the ability of 3 and 15 μg/ml of azithromycin to limit the replication of L. major was statistically equal to 1 mg/ml of amphotericin B. (FIG. 1).

Conclusion

The present invention shows that azithromycin has in vitro activity against parasites of the genus Leishmania. The results demonstrated a dose dependent decrease in the parasite counts and multiplication indices when azithromycin was used as the antibiotic. It is important to note that the 50% effective dose (ED₅₀) in these experiments was <0.6 μg/ml (Table 2 and FIG. 1), a concentration that can be readily achieved in human serum with oral dosing (Foulds G. et al., Journal of Antimicrobial Chemotherapy. 25 Suppl A:73-82, 1990). The measurement of azithromycin concentrations in serum after oral administration or in culture fluid in in vitro systems does not reflect accurate concentrations of the drug in the microenvironment of the pathogen since the drug has been shown to concentrate intracellularly. Therefore, in vitro models do not demonstrate the full potential of obtainable serum levels and concentrations of antibiotics like azithromycin due to the significant tissue penetration capabilities of azithromycin. (Donowitz G R. Clinical Infectious Diseases. 19(5):926-30, 1994).

TABLE 2
Effect of two doses of azithromycin (0.6 μg and 3.0 μg) administered at
two volumes (3 ml or 50 ml) on the ability of Leishmania major to infect
and multiply in murine macrophages maintained in vitro for five days.
Results presented are: (1) the total number of parasites found per 100
macrophages and (2) the percentage of macrophages infected without
regard to the number of parasites found per cell. Numbers in bold
represent 50% or greater reduction from control.

| | | | Number |
|---|---|---|---|
| | | Percent Infected | of Amastigotes/ |
| Treatment | Volume | Macrophages | 100 macrophages |
| Infection at time 0 | | 16 | 27 |
| Control | — | 29 | 35 |
| 0.6 μg azithromycin | 3 ml | 17 | 23 |
| 0.6 μg azithromycin | 50 ml | 10 | 11 |
| 3.0 μg azithromycin | 3 ml | 9 | 11 |
| 3.0 μg azithromycin | 50 ml | 11 | 13 |

Increasing the net amount of antibiotic in the culture medium by increasing the volume of the culture medium will affect parasite viability. The present invention demonstrates that increasing the volume of the culture medium at a single concentration of drug increases the efficacy of azithromycin against L. major. It is hypothesized that azithromycin was preferentially transported inside of the macrophage leading to enhanced activity in the presence of increased intracellular quantities of the drug.

The use of monocyte-derived bone marrow macrophages for drug susceptibility tests against Leishmania has not been reported before. The advantage of using these cells rather than peritoneal macrophages resides in the higher yield of cells per mouse. Approximately 50×106 monocyte-derived bone marrow macrophages are derived from a single mouse whereas only 0.7 to 1.2×10⁶ macrophages are recovered from the peritoneal cavity. Previous studies have demonstrated that the susceptibility of Leishmania sp. to different drugs is variable and dependent on the source of the macrophages. Specifically, it has been shown that Leishmania sp. are susceptible to pentavalent antimony in vivo and in vitro in mouse peritoneal macrophages and in human monocyte-derived macrophages, but not in tumor macrophages (Berman J D. & Wyler D J. Journal of Infectious Diseases. 142(1):83-6, 1980; Mattock, N. M. and Peters, W., Annals of Tropical Medicine & Parasitology, 69:359-71, 1975; Neal, R. A. and Matthews, P. J., Transactions of the Royal Society of Tropical Medicine & Hygiene, 76:284, 1982). Monocyte-derived bone marrow macrophages therefore are an alternative source for in vitro testing of drugs for efficacy against Leishmania sp.

Amphotericin B was used as a positive control in these studies as it has been previously reported to control Leishmania tropica and Leishmania donovani infections in human monocyte-derived macrophages cultures. The concentration used in the present invention followed previous reports and was based on the approximate plasma levels in humans treated with the drug (Berman J D. & Wyler D J, Journal of Infectious Diseases. 142(1):83-6, 1980). The results obtained with azithromycin were, however, comparable to those achieved in vitro with antimony gluconate and pentamidine tested in a similar system. These two drugs are effective and clinically indicated for therapy of leishmaniasis. (Berman J D. & Lee L S. Journal of Parasitology. 70(2):220-5, 1984., Berman J D. & Wyler D J. Journal of Infectious Diseases. 142(1):83-6, 1980).

The mechanism by which azithromycin controls L. major infection of macrophages is unknown. There is evidence that azithromycin has a direct effect on the survival of T. gondii by the inhibition of protein synthesis (Blais, J., et al., Antimicrobial Agents & Chemotherapy, 37:1701-3, 1993). Alternatively, azithromycin has been shown to enhance the ability of macrophages to eliminate a number of different pathogens (Bermudez, L. E., et al., Journal of Infectious Diseases, 169:575-80, 1994; Ouadrhiri, Y., et al, Antimicrobial Agents & Chemotherapy, 43: 1242-52, 1999; Xu, G., et al, Microbiology & Immunology, 40:473-9, 1996). If azithromycin operates by enhancing the killing capacity of macrophages, it would then be predicted that the efficacy of azithromycin in vivo would be greater than in vitro as the macrophages would be in the optimal physiological environment. Finally, it is possible that azithromycin has both a direct killing effect and an immunomodulatory effect which would yield optimal control of the parasites.

The present invention uses a novel source of macrophages for drug susceptibility tests against Leishmania, more specifically monocyte-derived bone marrow macrophages are used to determine the efficacy of anti-parasitic activity. The advantage of using monocyte-derived bone marrow macrophages rather than peritoneal macrophages resides in the higher yield of cells per mouse (50 million vs. 0.7 to 1.2 million cells per mouse).

In summary, azithromycin has activity against Leishmania major in an in vitro system at concentrations that are readily achieved in human serum. Given the severity of the problem, the paucity of new drugs and the limitations of those drugs that are available, therapeutic regimens using azithromycin will have a strong impact in the treatment protocols for leishmaniasis.

What is claimed is:

1. A method of treating a Leishmania sp. parasitic infection in a mammal, comprising administering a therapeutically effective amount of azithromycin to a patient in an amount determined to achieve sufficient serum concentrations of said azithromycin.

2. The method of claim 1, wherein said Leishmania sp. causes an opportunistic infection of leishmaniasis in patients with acquired immunodeficiency syndrome (AIDS).

3. The method of claim 1, wherein said azithromycin is administered orally, by injection, or intravenously.

4. A method of treating a Leishmania sp. parasitic infection in a mammal, comprising administering a therapeutically effective amount of azithromycin to a patient in an amount determined to achieve sufficient serum concentrations of said azithromycin, wherein said azithromycin accumulates in a phago-lysosome inside a macrophage.

5. A method of testing the susceptibility of a Leishmania sp. to a drug in vitro, comprising:
a) culturing amastigotes with monocyte-derived bone marrow macrophages;
b) adding concentrations of an antibiotic to be tested; and
c) measuring levels of macrophage infection by said amastigotes, wherein a number of infected macrophages and a number of said amastigotes in said infected macrophages are counted.

6. The method of claim 5, wherein said antibiotic is an azithromycin.

7. A method of preventing a Leishmania sp. parasitic infection in a mammal, comprising administering a prophylactically effective amount of azithromycin to a patient in an amount determined to achieve sufficient serum concentrations of said azithromycin, wherein said azithromycin accumulates in a phago-lysosome inside a macrophage.

8. The method of claim 7, wherein said azithromycin is administered is administered orally, by injection, or intravenously.
